(19) United States
(12) Patent Application Publication
(10) Pub. No.: US 2017/0368540 A1
(43) Pub. Date: Dec. 28, 2017

(54) ISOPARAFFIN-OLEFIN ALKYLATION

(71) Applicant: ExxonMobil Research and Engineering Company, Annandale, NJ (US)

(72) Inventors: Matthew S. METTLER, Somerville, NJ (US); Jihad M. DAKKA, Whitehouse Station, NJ (US); Ivy D. JOHNSON, Lawrenceville, NJ (US); Stefani PRIGOZHINA, Coatesville, PA (US); Charles M. SMITH, Princeton, NJ (US); William W. LONERGAN, Humble, TX (US); Brett LOVELESS, Houston, TX (US); Christine N. ELIA, Bridgewater, NJ (US); Wenyih F. LAI, Bridgewater, NJ (US)

(21) Appl. No.: 15/610,700

(22) Filed: Jun. 1, 2017

Related U.S. Application Data
(60) Provisional application No. 62/431,983, filed on Dec. 9, 2016.
(60) Provisional application No. 62/353,666, filed on Jun. 23, 2016.

(51) Int. Cl. B01J 29/70; C07C 2/62; B01J 29/035; B01J 29/18

(52) U.S. Cl. CPC B01J 29/7038; B01J 29/18; C07C 2/62; B01J 29/035; C07C 2521/04; C07C 2521/08; C07C 2529/035; C07C 2529/18; C07C 2521/06

(57) ABSTRACT

In a process for the catalytic alkylation of an olefin with an isoparaffi, an olefin-containing feed is contacted with an isoparaffin-containing feed under alkylation conditions in the presence of a solid acid catalyst comprising a crystalline microporous material of at least one of the MWW and MOR framework types, wherein the solid acid catalyst is substantially free of amorphous alumina.

### CROSS-REFERENCE TO RELATED APPLICATIONS

This application claims the benefit of U.S. Provisional Application No. 62/431,983, filed on Dec. 9, 2016, and U.S. Provisional Application No. 62/353,666, filed on Jun. 23, 2016, the entire contents of each are incorporated herein by reference.

### FIELD

The present disclosure relates to a process for isoparaffin-olefin alkylation.

### BACKGROUND

Alkylation is a reaction in which an alkyl group is added to an organic molecule. Thus an isoparaffin can be reacted with an olefin to provide an isoparaffin of higher molecular weight. Industrially, the concept depends on the reaction of a C₂ to C₅ olefin with isobutane in the presence of an acidic catalyst producing a so-called alkylate. This alkylate is a valuable blending component in the manufacture of gasoline due not only to its high octane rating but also to its sensitivity to octane-enhancing additives.

Industrial alkylation processes have historically used hydrofluoric or sulfuric acid catalysts under relatively low temperature conditions. The sulfuric acid alkylation reaction is particularly sensitive to temperature, with low temperatures being favored to minimize the side reaction of olefin polymerization. Acid strength in these liquid acid catalyzed alkylation processes is preferably maintained at 88 to 94 weight percent by the continuous addition of fresh acid and the continuous withdrawal of spent acid. The hydrofluoric acid process is less temperature sensitive and the acid is easily recovered and purified.

Both sulfuric acid and hydrofluoric acid alkylation share inherent drawbacks including environmental and safety concerns, acid consumption, and sludge disposal. Research efforts have been directed to developing alkylation catalysts which are equally as effective as sulfuric or hydrofluoric acids but which avoid many of the problems associated with these two acids. For a general discussion of sulfuric acid alkylation, see the series of three articles by L. F. Albright et al., “Alkylation of Isobutane with C₄ Olefins”, 27 Ind. Eng. Chem. Res., 381-397, (1988). For a survey of hydrofluoric acid catalyzed alkylation, see 1 Handbook of Petroleum Refining Processes 23-28 (R. A. Meyers, ed., 1986). A general overview of the technology can be found in “Chemistry, Catalysts and Processes of Isoparaffin-Olefin Alkylation—Actual Situation and Future Trends, Corma et al., Catal. Rev.—Sci. Eng. 35(4), 483-570 (1993).

With increasing demands for octane and increasing environmental concerns, it is desirable to develop an alkylation process employing safer, more environmentally acceptable catalyst systems. Specifically, it is desirable to provide an industrially viable alternative to the currently used hydrofluoric and sulfuric acid alkylation processes. Consequently, substantial efforts have been made to develop a viable isoparaffin-olefin alkylation process which avoids the environmental and safety problems associated with sulfuric and hydrofluoric acid alkylation while retaining the alkylate quality and reliability characteristics of these well-known processes. Research efforts have therefore for some time been directed towards solid, instead of liquid, alkylation catalyst systems.

For example, U.S. Pat. No. 3,644,565 discloses alkylation of a paraffin with an olefin in the presence of a catalyst comprising a Group VIII noble metal present on a crystalline aluminosilicate zeolite having pores of substantially uniform diameter from about 4 to 18 angstrom units and a silica to alumina ratio of 2.5 to 10, such as zeolite Y. The catalyst is pretreated with hydrogen to promote selectivity.

However, the development of a satisfactory solid acid replacement for hydrofluoric and sulfuric acid has proved challenging. For example, U.S. Pat. No. 4,384,161 describes a process of alkylating isoparaffins with olefins to provide alkylate using a large-pore zeolite catalyst capable of absorbing 2,2,4-trimethylpentane, for example, ZSM-4, ZSM-20, ZSM-3, ZSM-18, zeolite Beta, faujasite, mordenite, zeolite Y and the rare earth metal-containing forms thereof, and a Lewis acid such as boron trifluoride, antimony pentafluoride or aluminum trichloride. The addition of a Lewis acid is reported to increase the activity and selectivity of the zeolite, thereby effecting alkylation with high olefin space velocity and low isoparaffin/olefin ratio. According to the '161 patent, problems arise in the use of solid catalysts alone in that they appear to age rapidly and cannot perform effectively at high olefin space velocity.

As new solid acid catalysts have become available, they have been routinely screened for their efficacy in isoparaffin-olefin alkylation. For example, U.S. Pat. No. 5,304,698 describes a process for the catalytic alkylation of an olefin with an isoparaffin comprising contacting an olefin-containing feed with an isoparaffin-containing feed with a crystalline microporous material selected from the group consisting of MCM-22, MCM-36, and MCM-49 under alkylation conversion conditions of temperature at least equal to the critical temperature of the principal isoparaffin component of the feed and pressure at least equal to the critical pressure of the principal isoparaffin component of the feed.

Despite these advances, there remains a need for an improved isoparaffin-olefin alkylation process that is catalyzed by a solid acid catalyst but approaches or exceeds the activity and product quality of existing liquid phase processes.

### SUMMARY

According to the present disclosure, it has now been found that, by reducing or eliminating the alumina conventionally employed as a binder, the activity of MWW framework-type catalysts and MOR framework-type catalysts for isoparaffin-olefin alkylation can be significantly increased, in some cases by an amount approaching or exceeding 100%. This is surprising since, for most reactions, the activity of alumina-bound catalysts exceeds that of silica-bound or unbound catalysts (see, for example, U.S. Pat. No. 5,053,374).

Thus, in one aspect, the present disclosure provides a process for the catalytic alkylation of an olefin with an isoparaffin comprising, the process comprising: contacting an olefin-containing feed with an isoparaffin-containing feed under alkylation conditions in the presence of a solid acid catalyst comprising a crystalline microporous material of at least one of the MWW and MOR framework types, wherein the solid acid catalyst is substantially free of a binder containing amorphous alumina.

In a further aspect, the present disclosure provides a process for increasing olefin conversion in the catalytic alkylation of an olefin with an isoparaffin, the process comprising contacting an olefin-containing feed with an isoparaffin-containing feed under alkylation conditions in the presence of a solid acid catalyst comprising a crystalline microporous material of at least one of the MWW and MOR framework types, wherein the solid acid catalyst is substantially free of a binder containing amorphous alumina.

### BRIEF DESCRIPTION OF THE DRAWINGS

FIG. 1 is a graph of butane conversion against alpha activity for the catalysts of Examples 1 to 4.

FIG. 2 is a graph of butane conversion against cumene activity for the catalysts of Examples 1 to 4.

FIG. 3 is a graph of butane conversion against alpha activity for the catalysts of Examples 1, 4, and 5.

FIG. 4 is a graph of butane conversion against cumene activity for the catalysts of Examples 1, 4, and 5.

### DETAILED DESCRIPTION OF THE EMBODIMENTS

Disclosed herein is a process for isoparaffin-olefin alkylation, in which an olefin-containing feed is contacted with an isoparaffin-containing feed under alkylation conditions in the presence of a solid acid catalyst which comprises a crystalline microporous material of at least one of the MWW and MOR framework types and which is substantially free of any binder containing amorphous alumina.

As used herein, the term “crystalline microporous material of the MWW framework type” includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Crystalline microporous materials of the MWW framework type include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Examples of crystalline microporous materials of the MWW framework type include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO97/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), UZM-8HS (described in U.S. Pat. No. 7,713,513), UZM-37 (described in U.S. Pat. No. 7,982,084; EMM-10 (described in U.S. Pat. No. 7,842,277), EMM-12 (described in U.S. Pat. No. 8,704,025), EMM-13 (described in U.S. Pat. No. 8,704,023), MIT-1 (described by Luo et al in Chem. Sci., 2015, 6, 6320-6324), and mixtures thereof, with MCM-49 generally being preferred.

In some embodiments, the crystalline microporous material of the MWW framework type employed herein may be an aluminosilicate material having a silica to alumina molar ratio of at least 10, such as at least 10 to less than 50.

In some embodiments, the crystalline microporous material of the MWW framework type employed herein may be contaminated with other crystalline materials, such as ferrierite or quartz. These contaminants may be present in quantities <10% by weight, normally <5% by weight.

Also useful in the solid acid catalyst employed in the present process are crystalline microporous materials of the MOR framework type, including both naturally-occurring forms of mordenite as well as synthetic variants, such as TEA-mordenite.

As used herein, the term “substantially free of any binder containing amorphous alumina” means that the solid acid catalyst used herein contains less than 5 wt %, such as less than 1 wt %, and preferably no measurable amount, of amorphous alumina, typically used as a binder. Surprisingly, it is found that when the solid acid catalyst is substantially free of any amorphous alumina, the activity of the catalyst for isoparaffin-olefin alkylation can be significantly increased, for example by at least 50%, such as at least 75%, even at least 100% as compared with the activity of an identical catalyst but with an amorphous alumina binder. This result is illustrated in the subsequent Examples.

Other binder materials, including other inorganic oxides than alumina, such as silica, titania, zirconia and mixtures and compounds thereof, may be present in the solid acid catalyst used herein in amounts up to 90 wt %, for example up 80 wt %, such as up to 70 wt %, for example up to 60 wt %, such as up to 50 wt %. Where a non-alumina binder is present, the amount employed may be as little as 1 wt %, such as at least 5 wt %, for example at least 10 wt %. In one embodiment, a silica binder is employed such as disclosed in U.S. Pat. No. 5,053,374, the entire contents of which are incorporated herein by reference. In other embodiments, a zirconia or titania binder is used as described in the Examples.

In other embodiments, the crystalline microporous material is self-bound, that is substantially free of any inorganic oxide binder, although in some cases a temporary organic binder may be added to assist in forming the catalyst into the required shape. In such cases, the binder may be removed, such as by heating, before the catalyst is employed in the present alkylation process.

In other embodiments, the binder may be a crystalline oxide material such as the zeolite-bound-zeolites described in U.S. Pat. Nos. 5,665,325 and 5,993,642, the entire contents of which are incorporated herein by reference. In the case of crystalline binders, the binder material may contain alumina.

Feedstocks useful in the present alkylation process include at least one isoparaffin and at least one olefin. The isoparaffin reactant used in the present alkylation process may have from about 4 to about 8 carbon atoms. Representative examples of such isoparaffins include isobutane, isopentane, 3-methylhexane, 2-methylhexane, 2,3-dimethylbutane, 2,4-dimethylhexane and mixtures thereof, especially isobutane.

The olefin component of the feedstock may include at least one olefin having from 3 to 12 carbon atoms. Representative examples of such olefins include butene-2, isobutylene, butene-1, propylene, ethylene, hexene, octene, and heptene, merely to name a few. In some embodiments, the olefin component of the feedstock is selected from the group consisting of propylene, butenes, pentenes and mixtures thereof. For example, in one embodiment, the olefin component of the feedstock may include a mixture of propylene and at least one butene, especially 2-butene, where the weight ratio of propylene to butene is from 0.01:1 to 1.5:1, such as from 0.1:1 to 1:1. In another embodiment, the olefin component of the feedstock may include a mixture of propylene and at least one pentene, where the weight ratio of propylene to pentene is from 0.01:1 to 1.5:1, such as from 0.1:1 to 1:1.

Isoparaffin to olefin ratios in the reactor feed typically range from about 1.5:1 to about 100:1, such as 10:1 to 75:1, measured on a volume to volume basis, so as to produce a high quality alkylate product at industrially useful yields. Higher isoparaffin:olefin ratios may also be used, but limited availability of produced isoparaffin within many refineries coupled with the relatively high cost of purchased isoparaffin favor isoparaffin:olefin ratios within the ranges listed above.

Before being sent to the alkylation reactor, the isoparaffin and/or olefin may be treated to remove catalyst poisons e.g., using guard beds with specific absorbents for reducing the level of S, N, and/or oxygenates to values which do not affect catalyst stability activity and selectivity.

The present alkylation process is suitably conducted at temperatures from about 275° F. to about 700° F. (135° C. to 371° C.), such as from about 300° F. to about 600° F. (149° C. to 316° C.). Operating temperature typically exceed the critical temperature of the principal component in the feed. The term “principal component” as used herein is defined as the component of highest concentration in the feedstock. For example, isobutane is the principal component in a feedstock consisting of isobutane and 2-butene in isobutane:2-butene weight ratio of 50:1.

Operating pressure may similarly be controlled to maintain the principal component of the feed in the supercritical state, and is suitably from about 300 to about 1500 psig (2170 kPa-a to 10,445 kPa-a), such as from about 400 to about 1000 psig (2859 kPa-a to 6996 kPa-a). In some embodiments, the operating temperature and pressure remain above the critical value for the principal feed component during the entire process run, including the first contact between fresh catalyst and fresh feed.

Hydrocarbon flow through the alkylation zone containing the catalyst is typically controlled to provide an olefin liquid hourly space velocity (LHSV) sufficient to convert about 99 percent by weight of the fresh olefin to alkylate product. In some embodiments, olefin LHSV values fall within the range of about 0.01 to about 10 hr⁻¹.

The present isoparaffin-olefin alkylation process can be conducted in any known reactor, including reactors which allow for continuous or semi-continuous catalyst regeneration, such as fluidized and moving bed reactors, as well as swing bed reactor systems where multiple reactors are oscillated between on-stream mode and regeneration mode. Surprisingly, however, it is found that catalysts employing MWW framework type molecular sieves show unusual stability when used in isoparaffin-olefin alkylation. Thus, MWW-containing alkylation catalysts are particularly suitable for use in simple fixed bed reactors, without swing bed capability. In such cases, cycle lengths (on-stream times between successive catalyst regenerations) in excess of 150 days may be obtained.

The product composition of the isoparaffin-olefin alkylation reaction described herein is highly dependent on the reaction conditions and the composition of the olefin and isoparaffin feedstocks. In any event, the product is a complex mixture of hydrocarbons, since alkylation of the feed isoparaffin by the feed olefin is accompanied by a variety of competing reactions including cracking, olefin oligomerization and further alkylation of the alkylate product by the feed olefin. For example, in the case of alkylation of isobutane with C₃-C₅ olefins, particularly 2-butene, the product may comprise about 20 wt % of C₅-C₇ hydrocarbons, 60-65 wt % of octanes and 15-20 wt % of C₁₀+ hydrocarbons. Moreover, using an MWW type molecular sieve as the catalyst, it is found that the process is selective to desirable high octane components so that, in the case of alkylation of isobutane with C₃-C₅ olefins, the C₆ fraction typically comprises at least 40 wt %, such as at least 70 wt %, of 2,3-dimethylbutane, the C₇ fraction typically comprises at least 40 wt %, such as at least 80 wt %, of 2,3 dimethyl pentane and the C₈ fraction typically comprises at least 50 wt %, such as at least 70 wt %, of 2,3,4; 2,3,3 and 2,2,4-trimethylpentane.

The product of the isoparaffin-olefin alkylation reaction is conveniently fed to a separation system, such as a distillation train, to recover the C₈₋ fraction for use as a gasoline octane enhancer. Depending on alkylate demand, part of all of the remaining C₁₀₊ fraction can be recovered for use as a distillate blending stock or can be recycled to the alkylation reactor to generate more alkylate. In particular, it is found that MWW type molecular sieves are effective to crack the C₁₀₊ fraction to produce light olefins and paraffins which can react to generate additional alkylate product and thereby increase overall alkylate yield.

The invention will now be more particularly described with reference to the following non-limiting Examples and the accompanying drawings.

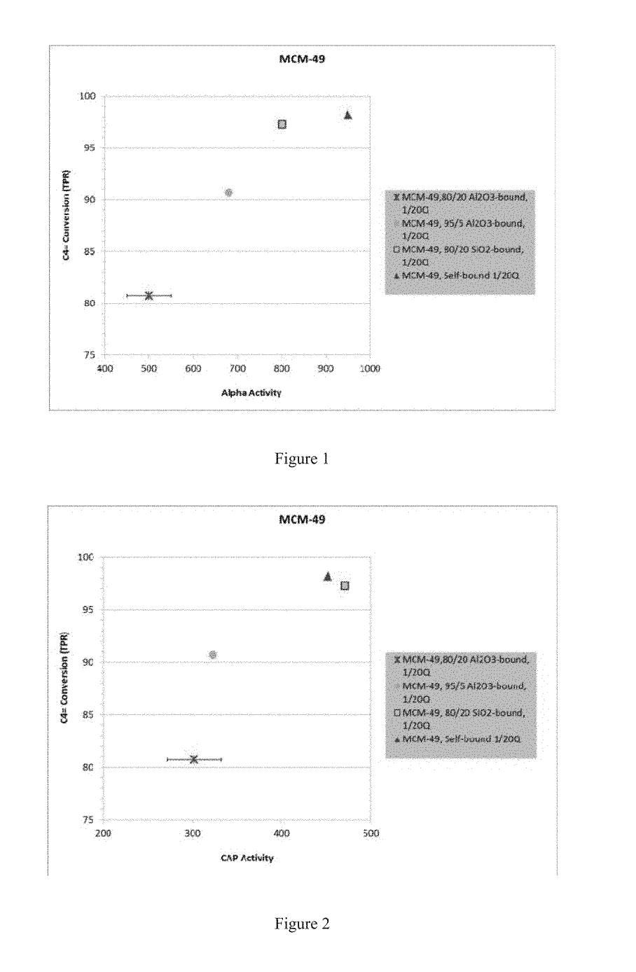

In the Examples, the following tests were used to measure the catalyst properties summarized in Tables 1 and 3 and FIGS. 1 and 2.

Alpha value is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538. ° C. and a variable flow rate as described in detail in the Journal of Catalysis, Vol. 61, p. 395

Cumene activity profile (CAP) assesses catalyst activity at the surface of a catalyst crystal. The reported values were determined according to the following procedure: Equipment

A 300 ml Parr batch reaction vessel equipped with a stir rod and static catalyst basket was used for the activity and selectivity measurements. The reaction vessel was fitted with two removable vessels for the introduction of benzene and propylene respectively.

### Feed Pretreatment

Benzene was obtained from a commercial source. The benzene was passed through a pretreatment vessel (2 L Hoke vessel) containing 500 cc. of molecular sieve 13X, followed by 500 cc. of molecular sieve 5 A, then 1000 cc. of Selexsorb CD, then 500 cc. of 80 wt. % MCM-49 and 20 wt. % Al₂O₃. All feed pretreatment materials were dried in a 260° C. oven for 12 hours before use.

Propylene was obtained from a commercial specialty gases source and was polymer grade. The propylene was passed through a 300 ml vessel containing pretreatment materials in the following order: (a) 150 ml molecular sieve 5 A and then (b) 150 ml Selexsorb CD. Both guard-bed materials were dried in a 260° C. oven for 12 hours before use.

Nitrogen was ultra high purity grade and obtained from a commercial specialty gases source. The nitrogen was passed through a 300 ml vessel containing pretreatment materials in the following order: (a) 150 ml molecular sieve 5 A and then (b) 150 ml Selexsorb CD. Both guard-bed materials were dried in a 260° C. oven for 12 hours before use.

### Catalyst Preparation and Loading

A 2 gram sample of catalyst was dried in an oven in air at 260° C. for 2 hours. The catalyst was removed from the oven and immediately 1 gram of catalyst was weighed. Quartz chips were used to line the bottom of a basket followed by loading of 0.5 or 1.0 gram of catalyst into the basket on top of the first layer of quartz. Quartz chips were then placed on top of the catalyst. The basket containing the catalyst and quartz chips was placed in an oven at 260° C. overnight in air for about 16 hours. The basket containing the catalyst and quartz chips was removed from the oven and immediately placed in the reactor and the reactor was immediately assembled.

### Test Sequence

The reactor temperature was set to 170° C. and purged with 100 sccm (standard cubic centimeter) of the ultra high purity nitrogen for 2 hours. After nitrogen purging the reactor for 2 hours, the reactor temperature was reduced to 130° C., the nitrogen purge was discontinued and the reactor vent closed. A 156.1 gram quantity of benzene was loaded into a 300 ml transfer vessel, performed in a closed system. The benzene vessel was pressurized to 2169 kPa-a (300 psig) with the ultra high purity nitrogen and the benzene was transferred into the reactor. The agitator speed was set to 500 rpm and the reactor was allowed to equilibrate for 1 hour. A 75 ml Hoke transfer vessel was then filled with 28.1 grams of liquid propylene and connected to the reactor vessel, and then connected with 2169 kPa-a (300 psig) ultra high purity nitrogen. After the one-hour benzene stir time had elapsed, the propylene was transferred from the Hoke vessel to the reactor. The 2169 kPa-a (300 psig) nitrogen source was maintained connected to the propylene vessel and open to the reactor during the entire run to maintain constant reaction pressure during the test. Liquid product samples were taken at 30, 60, 90, 120, and 180 minutes after addition of the propylene.

In the Examples below, selectivity is the weight ratio of recovered product diisopropylbenzene to recovered product isopropylbenzene (DIPB/IPB) after propylene conversion reached 99+%. The activity of all examples is determined by calculating the 2nd order rate constant for a batch reactor using mathematical techniques known to those skilled in the art.

### Example 1 Preparation of 80 wt % MCM-49/20 wt % Alumina Catalyst

80 parts MCM-49 zeolite crystals are combined with 20 parts pseudoboehmite alumina, on a calcined dry weight basis. The MCM-49 and pseudoboehmite alumina dry powder are placed in a muller or a mixer and mixed for about 10 to 30 minutes. Sufficient water and 0.05% polyvinyl alcohol are added to the MCM-49 and alumina during the mixing process to produce an extrudable paste. The extrudable paste is formed into a 1/20th inch quadralobe extrudate using an extruder. After extrusion, the 1/20th inch quadralobe extrudate is dried at a temperature ranging from 250° F. to 325° F. (121 to 163° C.). After drying, the dried extrudate is heated to 1000° F. (538° C.) under flowing nitrogen. The extrudate is then cooled to ambient temperature and humidified with saturated air or steam.

After humidification, the extrudate is ion exchanged with 0.5 to 1 N ammonium nitrate solution. The ammonium nitrate solution ion exchange is repeated. The ammonium nitrate exchanged extrudate is then washed with deionized water to remove residual nitrate prior to calcination in air. After washing the wet extrudate, it is dried. The exchanged and dried extrudate is then calcined in a nitrogen/air mixture to a temperature 1000′F (538° C.).

The properties of the resultant catalyst are summarized in Tables 1 and 2 and FIGS. 1 and 2.

### Example 2 Preparation of 95 wt % MCM-49/5 wt % Alumina Catalyst

95 parts MCM-49 zeolite crystals are combined with 5 parts pseudoboehmite alumina, on a calcined dry weight basis. The MCM-49 and pseudoboehmite alumina dry powder is placed in a muller or a mixer and mixed for about 3 to 30 minutes. Sufficient water and 0.05% polyvinyl alcohol is added to the MCM-49 and alumina during the mixing process to produce an extrudable paste. The extrudable paste is formed into a 1/20 inch quadralobe extrudate using an extruder. After extrusion, the 1/20th inch quadralobe extrudate is dried at a temperature ranging from 250° F. to 325° F. (121 to 163° C.). After drying, the dried extrudate is heated to 1000° F. (538° C.) under flowing nitrogen. The extrudate is then cooled to ambient temperature and humidified with saturated air or steam.

After humidification, the extrudate is ion exchanged with 0.5 to 1 N ammonium nitrate solution. The ammonium nitrate solution ion exchange is repeated. The ammonium nitrate exchanged extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.).

The properties of the resultant catalyst are summarized in Tables 1 and 2 and FIGS. 1 and 2.

### Example 3 Preparation of 80 wt % MCM-49/20 wt % Silica Catalyst

80 parts MCM-49 zeolite crystals are combined with 20 parts silica (Ultrasil and Ludox HS40), on a calcined dry weight basis. Sufficient water is added to the MCM-49 and silica during the mixing process to produce an extrudable paste. The extrudable paste is formed into a 1/20 inch extrudate using an extruder. After extrusion, the extrudate is dried at a temperature ranging from 250° F. to 325° F. (121 to 163° C.). After drying, the dried extrudate is heated to 1000° F. (538° C.) under flowing nitrogen. The extrudate is ion exchanged with 0.5 to 1 N ammonium nitrate solution. The exchanged and dried extrudate is then calcined in a nitrogen/air mixture to a temperature 1000° F. (538° C.).

The properties of the resultant catalyst are summarized in Tables 1 and 2 and FIGS. 1 and 2.

### Example 4 Preparation of 80 wt % MCM-49/20 wt % Zirconia Catalyst

80 parts MCM-49 zeolite crystals are combined with 20 parts zirconium oxide (Sigma-aldrich), on a calcined dry weight basis. The MCM-49 and ZrO₂ powder are placed in a muller or mixer and mixed for about 5 to 30 minutes. Sufficient water is added to the MCM-49 and silica during the mixing process to produce an extrudable paste. The extrudable paste is formed into a 1/20th inch extrudate using an extruder. After extrusion, the extrudate is dried at a temperature ranging from 250° F. to 325° F. (121 to 163° C.). After drying, the dried extrudate is heated to 1000° F. (538° C.) under flowing nitrogen. The extrudate is then cooled to ambient temperature and humidified with saturated air or steam.

The extrudate is ion exchanged with 0.5 to 1 N ammonium nitrate solution. The ammonium nitrate solution ion exchange is repeated. The ammonium nitrate extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.).

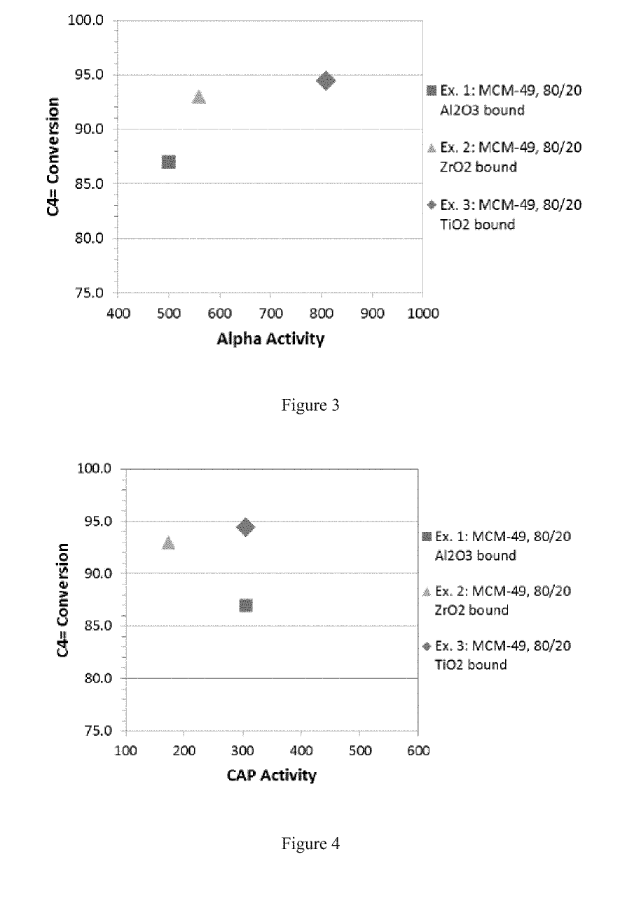

The properties of the resultant catalyst are summarized in Tables 1 and 2 and FIGS. 3 and 4.

### Example 5 Preparation of 80 wt % MCM-49/20 wt % Titania Catalyst

80 parts MCM-49 zeolite crystals are combined with 20 parts titanium oxide (Degussa P-25), on a calcined dry weight basis. The MCM-49 and ZrO₂ powder are placed in a muller or mixer and mixed for about 5 to 30 minutes. Sufficient water and 0.05% polyvinyl alcohol is added to the MCM-49 and silica during the mixing process to produce an extrudable paste. The extrudable paste is formed into a 1/20th inch extrudate using an extruder. After extrusion, the extrudate is dried at a temperature ranging from 250° F. to 325° F. (121 to 163° C.). After drying, the dried extrudate is heated to 1000° F. (538° C.) under flowing nitrogen. The extrudate is then cooled to ambient temperature and humidified with saturated air or steam.

The extrudate is ion exchanged with 0.5 to 1 N ammonium nitrate solution. The ammonium nitrate solution ion exchange is repeated. The ammonium nitrate extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.).

The properties of the resultant catalyst are summarized in Tables 1 and 2 and FIGS. 3 and 4.

### Example 6 Preparation of Self-Bound MCM-49 Catalyst

300 g MCM-49 zeolite crystals, on a calcined dry weight basis and 15 g Abitec Sterotex bioadditive are combined in the muller and mulled for 5 minutes. To the crystal. 280 g of water are added and mulling was continued for 5 minutes. An additional 300 g of MCM-49 crystal (calcined dry weight) and 15 g Albitec Sterotex bioadditive was gradually added to the mull mix and mulling continued for 10 minutes. An additional 500 g of water was added to form paste. The extrudable paste is formed into a 1/20th inch quadralobe extrudate using an extruder. After extrusion, the 1/20th inch quadralobe extrudate is dried at a temperature ranging from 250° F. to 325° F. (121 to 163° C.). The extrudate is then cooled to ambient temperature and humidified with saturated air or steam.

After humidification, the extrudate is ion exchanged with 0.5 to 1 N ammonium nitrate solution. The ammonium nitrate solution ion exchange is repeated. The ammonium nitrate exchanged extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.).

The properties of the resultant catalyst are summarized in Tables 1 and 2 and FIGS. 1 and 2.

### Example 7: Preparation of 65/35 wt % of Mordenite/Versal-300 Alumina Catalyst

A catalyst was made from a mixture of 65 parts (basis: calcined 538° C.) of mordenite crystals and 35 parts of Versal-300 alumina (basis: calcined 538° C.) in a muller. The mordenite crystals were first mulled in a muller for 5 minutes, then the Versal-300 alumina dry powder was added, and the mixture mulled for another 10 minutes. Water was added to the mixture of mordenite and alumina over a 5 minute period to the muller. The extrudable mixture was formed into a 1/16″ quadralobe extrudate using an extruder. After extrusion, the 1/16″ quadralobe extrudate was dried at 250° F. (121° C.). After drying, the dried extrudate was pre-calcined at 1000° F. (538° C.) in nitrogen. The pre-calcined extrudates were then cooled to ambient temperature and humidified with saturated air or steam. After humidification, the resulting extrudates were ion exchanged with 0.5N ammonium nitrate solution. The exchanged extrudates were then washed with deionized water to remove residual nitrate prior to drying and final calcination in air. The exchanged extrudate was dried at 121° C. and calcined in air at 538° C. The properties of the resultant catalyst are summarized in Tables 1 and 2.

### Example 8: Preparation of 65/35 wt % of Mordenite/Silica Catalyst

65 parts mordenite zeolite crystals are combined with 35 parts silica (Ultrasil and Ludox HS40), on a calcined dry weight basis. Sufficient water is added to the mordenite and silica during the mixing process to produce an extrudable paste. The extrudable paste is formed into a 1/16 inch extrudate using an extruder. After extrusion, the extrudate is dried at a temperature ranging from 250° F. to 325° F. (121 to 163° C.). After drying, the dried extrudate is heated to 1000° F. (538° C.) under flowing nitrogen. The extrudate is ion exchanged with 0.5 to 1 N ammonium nitrate solution. The exchanged and dried extrudate is then calcined in a nitrogen/air mixture to a temperature 1000° F. (538° C.). The properties of the resultant catalyst are summarized in Tables 1 and 2.

TABLE 1

| | | | Cumene Activity Profile | | | Alpha |
|---|---|---|---|---|---|---|
| | | (TPR) | | DIPB/IPB | TriPB/IPB | Hexane |
| Example | Description | C4 = conv. | activity | Selectivity | Selectivity | Cracking |
| Ex. 1 | MCM-49, 80/20 Al₂O₃, | 80.7 | 255 | 18.5 | 1.26 | 540 |
| Ex. 2 | MCM-49, 95/5 Al₂O₃ | 90.7 | 323 | 19.9 | 2.25 | 680 |
| Ex. 3 | MCM-49, 80/20 SiO₂ | 97.3 | 471 | 28.1 | 5.61 | 800 |
| Ex. 4 | MCM-49. 80/20 ZrO₂ | 93.0 | 173 | 26.0 | 5.0 | 560 |
| Ex. 5 | MCM-49, 80/20 TiO₂ | 94.4 | 305 | 27.3 | 5.3 | 810 |
| Ex. 6 | MCM-49, Self-bound | 98.2 | 452 | 29.3 | 6.1 | 950 |
| Ex. 7 | Mordenite, 65/35 Al₂O₃ | 68.5 | Not available | | | 490 |
| Ex. 8 | Mordenite, 65/35 SiO₂ | 76.1 | Not available | | | 640 |

TABLE 2

| | Collidine | | NH4 | NH4 | | BET-Total | Micropore | External | |
|---|---|---|---|---|---|---|---|---|---|
| | uptake, | hexane | TPAD | TPAD | NH4 | Surface | (ZSA), | (MSA), | Micropore |
| Example | umol/g | uptake | meq/g | Peak C | meq/g/C | area, m2/g | m2/g | m2/g | Volume, cc/g |
| Ex. 1 | 110 | 84.4 | 0.804 | 282 | 0.00472 | 508 | 337 | 171 | 0.1389 |
| Ex. 2 | 74 | 92.6 | 1.160 | 423 | 0.00552 | 542 | 448 | 94 | 0.1786 |
| Ex. 3 | 87.5 | 85.6 | 1.103 | 417 | 0.00548 | 498 | 393 | 105 | 0.1606 |
| Ex. 4 | 74 | | | | | 457 | 380 | 76.9 | 0.152 |
| Ex. 5 | 105 | | | | | 468 | 388 | 79 | 0.156 |
| Ex. 6 | 102 | 108 | 1.308 | 418 | 0.00656 | 700 | 582 | 118 | 0.2328 |
| Ex. 7 | | | | | | 495 | 315 | 180 | |
| Ex. 8 | | | | | | 452 | 352 | 98 | |

### Example 7 Alkylation Testing

The catalysts of Examples 1 to 4 were used in alkylation testing of a mixture of isobutane and 2-butene having the following composition (by weight):

| | |
|---|---|
| 1-butene | 0.01% |
| Cis-2-butene | 1.25% |
| Trans-2-butene | 1.19% |
| Iso-C₄═ | 0.00% |
| Iso-butane | 97.37% |
| n-butane | 0.23% |

The reactor used in these experiments comprised a stainless steel tube having an internal diameter of ⅜ in, a length of 20.5 in and a wall thickness of 0.035 in. A piece of stainless steel tubing 8¾ in. long×⅜ in. external diameter and a piece of inch tubing of similar length were positioned in the bottom of the reactor (one inside of the other) as a spacer to position and support the catalyst in the isothermal zone of the furnace. A ¼ inch plug of glass wool was placed at the top of the spacer to keep the catalyst in place. A ⅛ inch stainless steel thermo-well was placed in the catalyst bed, long enough to monitor temperature throughout the catalyst bed using a movable thermocouple. The catalyst is loaded with a spacer at the bottom to keep the catalyst bed in the center of the furnace's isothermal zone.

The catalyst was then loaded into the reactor from the top. The catalyst bed typically contained about 4 gm of catalyst sized to 14-25 mesh (700 to 1400 micron) and was 10 cm. in length. A ¼ in. plug of glass wool was placed at the top of the catalyst bed to separate quartz chips from the catalyst. The remaining void space at the top of the reactor was filled with quartz chips. The reactor was installed in the furnace with the catalyst bed in the middle of the furnace at the pre-marked isothermal zone. The reactor was then pressure and leak tested typically at 300 psig (2170 kPa-a).

500 cc ISCO syringe pumps were used to introduce the feed to the reactor. Two ISCO pumps were used for pumping the iso-butane (high flow rate 10-250 cc/hr) and one ISCO pump for pumping 2-butene (0.1-5 cc/hr). A Grove “Mity Mite” back pressure controller was used to control the reactor pressure typically at 750 psig (5272 kPa-a). On-line GC analyses were taken to verify feed and the product composition. The feed was then pumped through the catalyst bed held at the reaction temperature of 150° C. The products exiting the reactor flowed through heated lines routed to GC then to three cold (5-7° C.) collection pots in series. The non-condensable gas products were routed through a gas pump for analyzing the gas effluent. Material balances were taken at 24 hr intervals. Samples were taken for analysis. The material balance and the gas samples were taken at the same time while an on-line GC analysis was conducted for doing material balance.

The results of the MWW catalyst screening tests are summarized in Table 3 and show, based on first order kinetics, that the 80/20 MCM-49/silica bound catalyst of Example 3 exhibited 85% higher activity than the base case, the 80/20 MCM-49/alumina bound catalyst of Example 1, whereas the self-bound catalyst of Example 4 exhibited 120% higher activity than the base case.

TABLE 3

| Example | Catalyst | Relative Activity |
|---|---|---|
| 1 | MCM-49 80/20 Al₂O₃ | 1.0 [Base Case] |
| 2 | MCM-49 95/5 Al₂O₃ | 1.3 |
| 3 | MCM-49 80/20 SiO₂ | 1.8 |
| 4 | MCM-49 (Self-Bound) | 2.2 |

The results of the mordenite catalyst screening test are summarized in Table 4

TABLE 4

| Example | Catalyst | Relative activity |
|---|---|---|
| 5 | Mordenite 65/35 Al₂O₃ | 1.0 [Base Case] |
| 6 | Mordenite 65/35 SiO₂ | 1.1 |

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for the catalytic alkylation of an olefin with an isoparaffin comprising, the process comprising: contacting an olefin-containing feed with an isoparaffin-containing feed under alkylation conditions in the presence of a solid acid catalyst comprising a crystalline microporous material of at least one of the MWW and MOR framework types, wherein the solid acid catalyst is substantially free of a binder containing amorphous alumina.

2. The process of claim 1, wherein the solid acid catalyst is substantially binder-free.

3. The process of claim 1, wherein the solid acid catalyst comprises a binder comprising a crystalline molecular sieve.

4. The process of claim 1, wherein the solid acid catalyst comprises at least one of a silica, titania, and zirconia binder.

5. The process of claim 1, wherein the solid acid catalyst comprises a crystalline microporous material of the MWW framework type.

6. The process of claim 5, wherein the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, MIT-1, and mixtures thereof.

7. The process of claim 5, wherein the crystalline microporous material of the MWW framework type comprises MCM-49.

8. The process of claim 5, wherein the MWW framework type material contains up to 10% by weight of impurities of other framework structures.

9. The process of claim 1, wherein the olefin-containing feed comprises at least one C₃ to Cu olefin.

10. The process of claim 1, wherein the olefin-containing feed is selected from the group consisting of propylene, butenes, pentenes and mixtures thereof.

11. The process of claim 1, wherein the isoparaffin-containing feed comprises at least one C₄ to C₈ isoparaffin.

12. The process of claim 1, wherein the isoparaffin-containing feed comprises isobutane.

13. The process of claim 12, wherein the contacting produces an alkylate product having a C₆ fraction comprising at least 40 wt % of 2,3-dimethylbutane.

14. The process of claim 13, wherein the C₆ fraction of the alkylate product comprises at least 70 wt % of 2,3-dimethylbutane.

15. The process of claim 1, wherein at least one of the olefin-containing feed and the isoparaffin-containing feed is pretreated to remove impurities prior to the contacting step.

16. The process of claim 1, wherein the alkylation conditions include a temperature at least equal to the critical temperature of the principal component of the combined olefin-containing feed and isoparaffin-containing feed and pressure at least equal to the critical pressure of the principal component of the combined olefin-containing feed and isoparaffin-containing feed.

17. A process for increasing olefin conversion in the catalytic alkylation of an olefin with an isoparaffin, the process comprising contacting an olefin-containing feed with an isoparaffin-containing feed under alkylation conditions in the presence of a solid acid catalyst comprising a crystalline microporous material of at least one of the MWW and MOR framework types, wherein the solid acid catalyst is substantially free of a binder containing amorphous alumina.

18. The process of claim 17, wherein the solid acid catalyst is substantially binder-free.

19. The process of claim 17, wherein the solid acid catalyst comprises a binder comprising a crystalline molecular sieve.

20. The process of claim 17, wherein the solid acid catalyst comprises at least one of a silica, titania, and zirconia binder.

21. The process of claim 17, wherein the solid acid catalyst comprises a crystalline microporous material of the MWW framework type.

22. The process of claim 17, wherein the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, MIT-1, and mixtures thereof.

23. The process of claim 17, wherein the olefin-containing feed comprises at least one C₃ to C₁₂ olefin.

24. The process of claim 17, wherein the olefin-containing feed is selected from the group consisting of propylene, butenes, pentenes and mixtures thereof.

25. The process of claim 17, wherein the isoparaffin-containing feed comprises at least one C₄ to C₈ isoparaffin.

26. The process of claim 17, wherein the isoparaffin-containing feed comprises isobutane.

27. The process of claim 26, wherein the contacting produces an alkylate product having a C₆ fraction comprising at least 40 wt % of 2,3-dimethylbutane.

28. The process of claim 26, wherein the C₆ fraction of the alkylate product comprises at least 70 wt % of 2,3-dimethylbutane.

29. The process of claim 17, wherein the alkylation conditions include a temperature at least equal to the critical temperature of the principal component of the combined olefin-containing feed and isoparaffin-containing feed and pressure at least equal to the critical pressure of the principal component of the combined olefin-containing feed and isoparaffin-containing feed.
